(19) United States
(12) Patent Application Publication
(10) Pub. No.: US 2022/0055237 A1
(43) Pub. Date: Feb. 24, 2022

(54) ELECTRIC SHAVER WITH LIGHT THERAPY

(71) Applicant: Peter Cordani, Jupiter, FL (US)

(72) Inventor: Peter Cordani, Jupiter, FL (US)

(21) Appl. No.: 17/406,160

(22) Filed: Aug. 19, 2021

Related U.S. Application Data
(60) Provisional application No. 63/067,357, filed on Aug. 19, 2020.

(51) Int. Cl. B26B 19/46; A61N 5/06; B26B 19/14

(52) U.S. Cl. CPC B26B 19/46; A61N 5/06; A61N 2005/0644; A61N 2005/0663; A61N 2005/0652; B26B 19/14

(57) ABSTRACT

A shaving device having a cutting head tuned for skin conditioning using light therapy. The cutting head of the shaving device includes a plurality of LED lights positioned around cutting blades which provide light therapy to the skin immediately after shaving using a range between 400 nm and 700 nm.

### PRIORITY CLAIM

In accordance with 37 C.F.R. § 1.76, a claim of priority is included in an Application Data Sheet filed concurrently herewith. Accordingly, the present invention claims priority to U.S. Provisional Patent Application No. 63/067,357, entitled “ELECTRIC SHAVER WITH LIGHT THERAPY”, filed Aug. 19, 2020. The contents of which the above referenced application is incorporated herein by reference.

### FIELD OF THE INVENTION

This invention is related to the field of hair removal and, in particular, to an electric shaver with LED light therapy.

### BACKGROUND OF THE INVENTION

It is commonly practice for men use to use shaving devices to remove unwanted hair from their face. Women may use such devices for removal of hair from their legs and arms. Depending on the individual, hair devices are difficult to tolerate on sensitive skin. Electric shavers, especially the popular shavers using three floating heads, are less abrasive on the skin. However, sensitivity is dependent upon the individual. In many instances, the mere cutting of hair follicles at a right angle to the skin surface can result in skin sensitivity as the follicles are sharp.

Electric shaving devices have two main components: a base in which a drive motor and battery reside, and the cutting head. The size and shape of the device allows for a variety of cutting head designs. Common to the most popular cutting head shape is a substantially triangular configuration employing three circular cutting blades that are spaced apart. The spaced apart blades allows for the closer tracking of an individual's bone structure to permit a more uniform and close shave. For instance, the triangular shape allows a man to cut hair between his cheek and neck by moving the shaver across the jaw bone to allow a cutting of the hair follicles close to the skin. Similarly improvement in shavers allow women to remove hair to a point it would not appear hair was ever present.

The problem with the improved shaving devices is that the hair removal is now so close to the skin, skin abrasion is the common result. Further, electric shavers generate heat and as the blades wear the heat and dull blade agitate the hair frolics. When the skin is damaged in any manner it is susceptible to premature aging. After shaving, whether the hair removal is from a man's face or the lady's legs, abrasion damaged can be lessened if moisturizer is applied to skin.

LED light therapy has been found to be beneficial for treating damaged, aging, and/or blemished skin. LED light therapy consists of positioning LED's of a particular color and intensity closely against the skin for a period of time. Generally, red lights are marketed to promote anti-aging properties within the skin; blue lights are marketed for reducing mild acne vulgaris.

What is lacking in the industry is a device incorporating LED light therapy for addressing skin irritation in combination with shaving, wherein the skin is treated at the moment skin irritation is caused by the act of shaving.

### SUMMARY OF THE INVENTION

Disclosed is a shaving device having a cutting head tuned for skin conditioning using light therapy. The cutting head of the shaving device includes a plurality of LED lights positioned around cutting blades to provide light therapy to the skin during shaving. The present invention overcomes the deficiencies of electric shavers and light therapy devices by combining the technology wherein the skin is treated instantly upon shaving. Strategic placement of LED lights within the shaving device provides skin healing light wavelengths to impact the skin at the same time the skin is damaged by the act of shaving. The shaver with light therapy provides skin-renewal.

A primary objective of this invention is to provide users with a level of convenience with a device that has the potential to reduce normal grooming routine times. This invention provides the ability to multitask by carrying out both the function of shaving and skin treatment with a single device that simultaneously allows for shaving and skin treatment.

Another objective of this invention is to provide a device for shaving and/or trimming of hair that employs the common electric shaver shape with modification only to the base of the cutting head.

Yet another objective of this device is to provide a device with LED lights embedded at a location directly over the desired area of skin for treatment using red wavelengths to treat skin aging and a blue wavelength to treat minor acne.

Another objective of the invention is to utilize the spacing between cutting blades of a shaver, thereby eliminating the need for shaver redesign.

Still another objective of the invention is use the existing shaver battery as a power source for light therapy lighting that is mounted on the shaver.

Another objective of the invention is to teach the use of light therapy on skin abrasion caused through the act of shaving.

Another objective is to teach the use of light therapy on shaving skin in the range of 400 nm to 700 nm.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE DRAWING

FIG. 1 presents a pictorial view of the shaving and light therapy device in a combined and completed state.

### DETAILED DESCRIPTION OF THE INVENTION

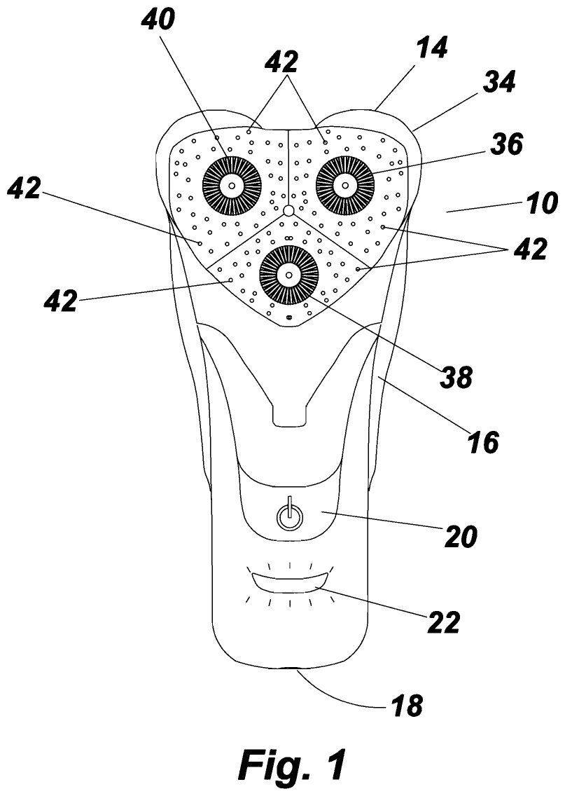

The following detailed description is merely representative in nature and does not limit the embodiments of the invention. Referring to FIG. 1, the electric shaving device 10 has a body portion 12 and a cutting head portion 14. The body portion 12 contains an ergonomically shaped sidewall 16 housing a rechargeable battery and motor, not shown. The battery is recharged by a power port 18 using an electrical power cord. Operation of the motor is by an on/off switch 20 with an operational mode provided by a light 22 to indicate an “on” position.

The cutting head portion 14 is defined by a substantially triangular base 34 securable to the body portion with three circular shaped cutting blades 36, 38 and 40 operatively associated with the base 34. In the preferred embodiment, the use of three blades provides a commonly accepted pattern, allowing ease of the razor use over a variety of skin configurations. For men, the problem with obtaining a smooth cut over the jaw bone area is pressing the shaver 10 against the skin can leave irritation. Skin irritation need not be due to facial or body structure, an individual may simply be sensitive to close shaves on the face, legs or under arms. Along the triangular shaped cutting head portion 34 are a plurality of LED lights 42 that are operative whenever the on/off switch 20 is placed in the operating position. In the preferred embodiment, miniature LED's are positioned in all the free space not consumed by the cutting blades 38. Once the shaver 10 is powered on, the user can hold the device using the body portion and the ergonomically designed upper grip. The electric shaving device 10 can then be used wherein the device's razor blades 36, 38, and 40 are used to shave off unwanted hair in desired areas. The battery, not shown, possesses the capability to enumerate the plurality of LED lights 42. Light therapy has been known to assist an individual in numerous ways. For instance, light therapy has been used to treat seasonable affective disorder (SAD) by exposure to bight light that mimics natural outdoor light. The beneficial healing light is known to stimulate the body's circulatory system, immune system, endocrine system and nervous system. Light is composed of waves and is measured in wavelengths. The beneficial healing effect of these wave frequencies is that they translate energy to the cells to repair the body. Colored light therapy can influence the health of both the physical body and the energetic body. Each color of the spectrum is considered to have its own nutritional value. The red end of the spectrum is considered to be more energizing, while the blue end of the spectrum is considered to be more restorative. The lights 42 provided can be colored per the user's desire. For instance, blue could be used to address acne reduction and red could be used to provide an anti-aging effect. In one embodiment the LED lights 42 are capable of transducing light in the range of 400-700 nm wavelength, and in particular a transducing red light at about 660 nm wavelength for skin restoration. Red light providing therapeutic application and to treat SAD. Transducing a blue light of about 415 nm is used for killing Propionibacterium acne or the bacteria associated with acne. During the time an individual is shaving the light treatment is conditioning the skin. It is noted that the skin is especially sensitive during the act of shaving, wherein the light therapy is most effective.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The use of the term “or” in the claims is used to mean “and/or” unless explicitly indicated to refer to alternatives only or the alternative are mutually exclusive, although the disclosure supports a definition that refers to only alternatives and “and/or.” The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Furthermore, a device or structure that is configured in a certain way is configured in at least that way, but may also be configured in ways that are not listed.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A shaving device comprising:
a body, said body housing an rechargeable battery coupled to an electric motor and at least one cutting head for removing hair from the surface of skin coupled to said electric motor; and a plurality of LED lights positioned around said cutting head, said LED lights producing a transducing light in the range of 500-700 nm wavelength; wherein operation of said cutting head for removing hair allowing simultaneous condition of the shaved skin with the transducing light.

2. The shaving device according to claim 1 where said cutting head is further defined by a substantially triangular hosing three circular shaped cutting blades.

3. The shaving device according to claim 1 where said LED lights produce transducing red light at about 660 nm wavelength.

4. The shaving device according to claim 1 where said LED lights produce transducing blue light at about 415 nm wavelength.
